(19) United States
(12) Patent Application Publication
(10) Pub. No.: US 2015/0164726 A1
(43) Pub. Date: Jun. 18, 2015

(54) AIRWAY SUPPORT DEVICE

(71) Applicant: THE REGENTS OF THE UNIVERSITY OF MICHIGAN, Ann Arbor, MI (US)

(72) Inventors: Kevin R. Ward, Superior Township, MI (US); Albert J. Shih, Ann Arbor, MI (US); Jeffrey Stephen Plott, Algonac, MI (US); Christopher Shih, Ann Arbor, MI (US)

(21) Appl. No.: 14/565,947

(22) Filed: Dec. 10, 2014

Related U.S. Application Data
(60) Provisional application No. 61/915,421, filed on Dec. 12, 2013.

(51) Int. Cl. A61G 13/12; A61M 16/00; A61M 16/06

(52) U.S. Cl. CPC A61G 13/1215; A61M 16/0683; A61M 16/0003; A61M 2016/0027; A61M 2202/0208; A61M 2205/3331; A61M 2230/04

(57) ABSTRACT

An airway support appliance lifts a patient's jaw upwards by using the mid-face of the patient as a stable fulcrum, thereby alleviating the need for techniques or devices to detect or alleviate airway obstructions. The appliance spans the patient's lower face and is secured to the angle and ramus of each side of the mobile mandible. These attachments are linked to one another across the midface or maxilla, using the bridge of the nose or the area between the nose and upper lip of the patient as the stabilizing point. The appliance may include an adjustment mechanism to facilitate tightening. Upon securement of the device to the patient while in a supine position, and activation of the adjustment mechanism, the mandible of the patient, which is mobile, is lifted anteriorly. Upon lifting of the mandible, the patient's tongue is carried forward and away from the posterior pharynx.

### CROSS-REFERENCE TO RELATED APPLICATIONS

This application is a non-provisional of U.S. Provisional Application No. 61/915,421 filed Dec. 12, 2013. This application claims the benefit of the filing date of U.S. Provisional Application No. 61/915,421 under 35 USC §119(e). U.S. Provisional Application No. 61/915,421 is hereby incorporated by reference.

### FIELD OF THE DISCLOSURE

This disclosure relates generally to devices to assist in maintaining a patient's airway and, more particularly, to an appliance securable to the lower face of a patient that uses the mid-face of the patient as a stable fulcrum to lift the patient's jaw upwards.

### BACKGROUND

Airway obstruction from a patient's posterior tongue surface contacting the anterior wall of the posterior pharynx can result in severe hypoxemia, brain damage or cardiac arrest. Airway obstructions can occur during a variety of traumatic and even typically atraumatic situations, including patients with brain injuries, patients with severe alcohol or drug intoxication, patients undergoing procedural sedation, such as during dental procedures, emergency department procedures, colonoscopies, minor surgery, and patients recovering from general anesthesia. Even patients with severe obstructive sleep apnea are victims of this type of airway obstruction. There are several techniques and devices to help detect or alleviate airway obstructions. These include: Close and substantially constant supervision by trained individuals examining chest rise and fall and other physiologic parameters; placement of nasopharyngeal or oropharyngeal airways upon the detection of patient whose airways appear to be actively obstructed; and providing a designated caregiver whose duties include applying a jaw thrust maneuver to the patient so as to actively maintain airway patency. Noninvasive ventilation devices such as bi-level positive airway pressure (BiPAP) are also used to overcome mechanical obstructions of the tongue.

These conventional approaches have associated drawbacks. For instance, monitoring chest rise and fall is labor intensive, may require long period of observation, and is susceptible to observer fatigue or human error. Placement of nasopharyngeal airway devices may cause epistaxis, which in turn could result in aspiration. Nasopharyngeal airway devices are also uncomfortable to place and are not well tolerated as patients regain consciousness. As to oropharyngeal airway devices, the patient must not have a gag reflex at the time of placement. However, as the patient regains consciousness, the device may trigger the gag reflex, which may result in vomiting, thus placing the patient at risk for aspiration and, potentially, death. Providing a caregiver to maintain the patient's open airway by applying the jaw thrust maneuver also requires constant monitoring, and, as this intervention technique is only performed reactively, it may undesirably permit at least some degree of potentially damaging airway obstruction. Use of a BiPAP-type device is expensive.

While there are a variety of commercial devices that purport to provide adequate jaw thrust or jaw elevation to maintain the patient's airway open during procedures or situation susceptible to airway blockage, it is our understanding that all such devices employ a behind-the-neck approach when performing the jaw thrusting procedure. In other words, these conventional devices either include a support that constrains the head from behind the neck, support the head from behind the neck, or require use in conjunction with a cervical collar that otherwise applies a constraint behind the head so as to support the jaw. A drawback of such behind-the-neck devices is that they require substantial repositioning of the patient, which movement in and of itself may aggravate the patient's condition. For example, any movement of the neck or head would be contraindicated for patients known or believed to have sustained severe neck or back trauma.

Additionally, performance of airway maneuvers such as bag-valve-mask (BVM) ventilation can be difficult, as it requires the performer to seal the mask around the patient's mouth and nose in addition to simultaneously performing a jaw thrust. The inability to perform an adequate jaw thrust in this situation may prevent the patient from receiving the desired ventilation from the bag. Similar issues occur when using a continuous positive airway pressure (CPAP) or a BiPAP mask for use in noninvasive ventilation in emergencies or for sleep apnea.

We are not aware of any existing airway support devices for use in patients undergoing procedural sedation or having an altered level of consciousness that do not suffer from any of the foregoing drawbacks.

### SUMMARY OF THE DISCLOSURE

An externally-worn appliance is disclosed that is capable of lifting the jaw upwards by using the mid-face of the patient as a stable fulcrum. The device may be adapted, for example through the use of integrated ports or clips, to work in concert with systems that provide oxygen to the patient and/or monitor levels of carbon dioxide. It may also be adapted to work with other sensors such as pulse oximetry as well as other tissue perfusion monitoring devices. The appliance of the present disclosure spans the patient's lower face and is secured to the angle and ramus of each side of the mobile mandible. Jaw-thrusting engagement pieces of the appliance are linked to one another across the midface or maxilla. In a first embodiment of the device, the bridge of the nose is used as the stabilizing point. In an alternate embodiment of the device, the area between the nose and upper lip of the patient is used as a stabilizing point. The device may be adapted to include an adjustment mechanism, which may take the form of springs, steps, or a ratchet mechanism (such as a gear rack and ratchet similar to a zip tie or cable tie), to allow for tightening or passive biasing of the jaw-thrusting engagement pieces toward the stabilizing point. Upon securement of the device to the patient while in a supine position, and activation (i.e., tightening) of the adjustment mechanism, the mandible of the patient, which is mobile, is lifted anteriorly. Upon lifting of the mandible, the patient's tongue is carried forward and away from the posterior pharynx.

### BRIEF DESCRIPTION OF THE SEVERAL VIEWS OF THE DRAWINGS

FIG. 1 is a perspective view of a first embodiment of an externally-worn airway support appliance of the present disclosure, as positioned on a patient;

FIG. 2 is a perspective view of the airway support appliance illustrated in FIG. 1;

FIG. 3 is a bottom view of the airway support appliance illustrated in FIG. 1;

FIG. 4 is a top perspective view of a second embodiment of an externally-worn airway support appliance of the present disclosure;

FIG. 4A is a front end view illustrating an interface of a track member and a connecting portion of a right reversible tension mechanism of the airway support appliance of FIG. 4, along the line 4A of FIG. 4;

FIG. 5 is a top perspective view of a right jaw-thrusting engagement piece and reversible tension mechanism of the airway support appliance illustrated in FIG. 4;

FIG. 6 is a rear perspective view of a left jaw-thrusting engagement piece of the airway support appliance illustrated in FIG. 1 or 4;

FIG. 7 is a rear perspective view of the right jaw-thrusting engagement piece of the airway support appliance illustrated in FIG. 1 or 4;

FIG. 8 is a top perspective view of the airway support appliance illustrated in FIG. 4, with the reversible tension mechanisms of the left and right jaw-thrusting engagement pieces advanced to locate the engagement surfaces of the left and right jaw-thrusting engagement pieces closer to the bridge support member of the airway support appliance;

FIG. 9 is a top perspective view of the airway support appliance illustrated in FIG. 4, with the reversible tension mechanisms of the left and right jaw-thrusting engagement pieces hingedly rotated to a collapsed condition relative to respective adjustment tracks of the bridge support member;

FIG. 10 is a top perspective view of the airway support appliance illustrated in FIG. 4, to which a nasal breathing tube is secured;

FIG. 11 is a top perspective view of an alternate embodiment of an airway support appliance of the present disclosure, with an aperture for accommodating an anesthesia mask;

FIG. 12 is a top perspective view of the airway support appliance of FIG. 11, with an anesthesia mask received in the aperture;

FIG. 13 is an exploded view of an airway support appliance as illustrated in FIG. 4, with an airway cradle of sufficient size to receive an anesthesia mask;

FIG. 14 is a top perspective view of the airway support appliance illustrated in FIG. 4, with an anesthesia mask received in the airway cradle; and

FIG. 15 is a top and left perspective view of an airway support appliance of the present disclosure having an alternate tension mechanisms of the left and right jaw-thrusting engagement pieces, each in the form of a spring;

### DETAILED DESCRIPTION OF THE PREFERRED EMBODIMENTS

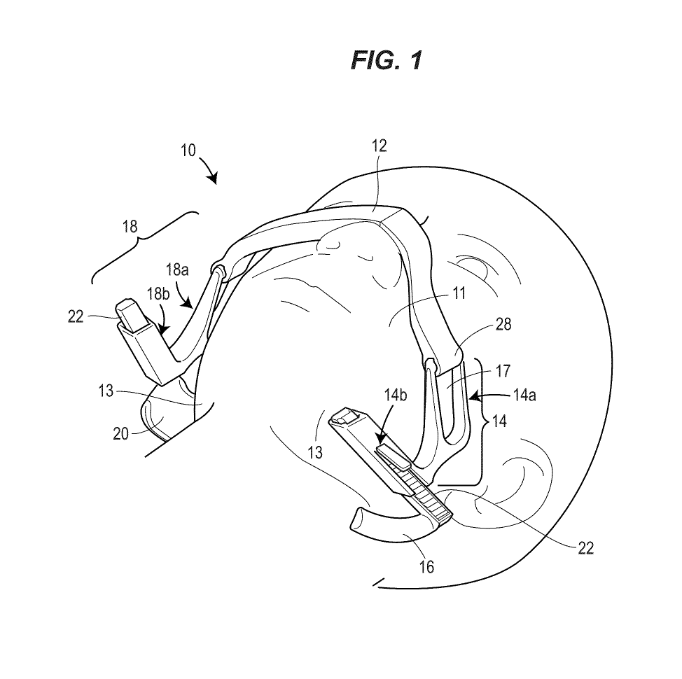

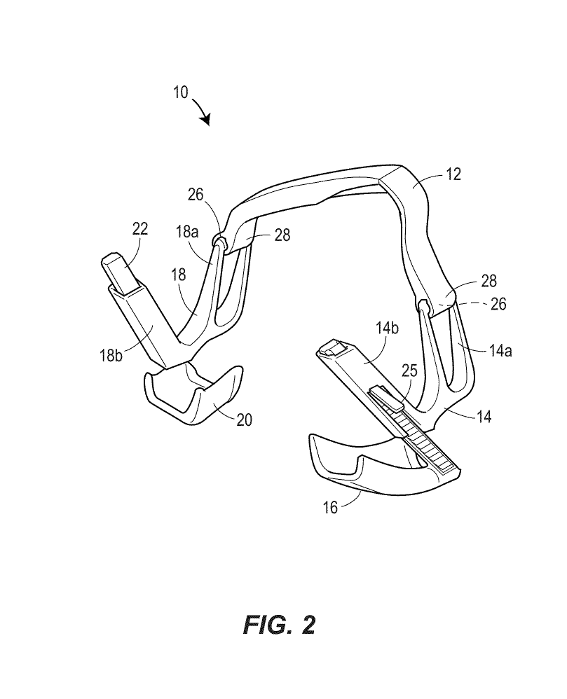

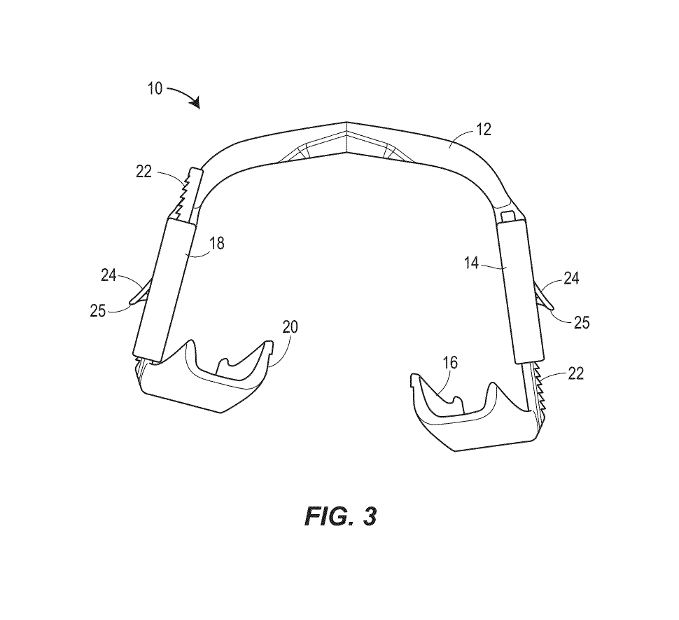

During jaw relaxation, such as during procedural sedation, there is the potential for the tongue of a patient to block the patient's airway, preventing airflow to the lungs. A jaw thrust maneuver counters airway blockage because by moving the jaw forward, the tongue is carried forward and away from the posterior pharynx. With reference to the drawings, an airway support appliance 10 spans the patient's lower face 11 and is secured to the angle and ramus of each side of the mobile mandible 13. In a first embodiment, illustrated in FIGS. 1-3, the airway support appliance 10 includes a bridge support member 12, a left hinge and reversible tension mechanism 14 (including a left hinge 14a and a left reversible tension mechanism 14b, which may be integral, as illustrated, or alternately, by way of example only, be made of separate left hinge 14a and left reversible tension mechanism 14b pieces that snap together or otherwise securely engage one another), a left jaw engagement piece 16, a right hinge and reversible tension mechanism 18 (including a right hinge 18a and a right reversible tension mechanism 18b, which may be integral, as illustrated, or alternately, by way of example only, be made of separate right hinge 18a and right reversible tension mechanism 18b pieces that snap together or otherwise securely engage one another), and a right jaw engagement piece 20.

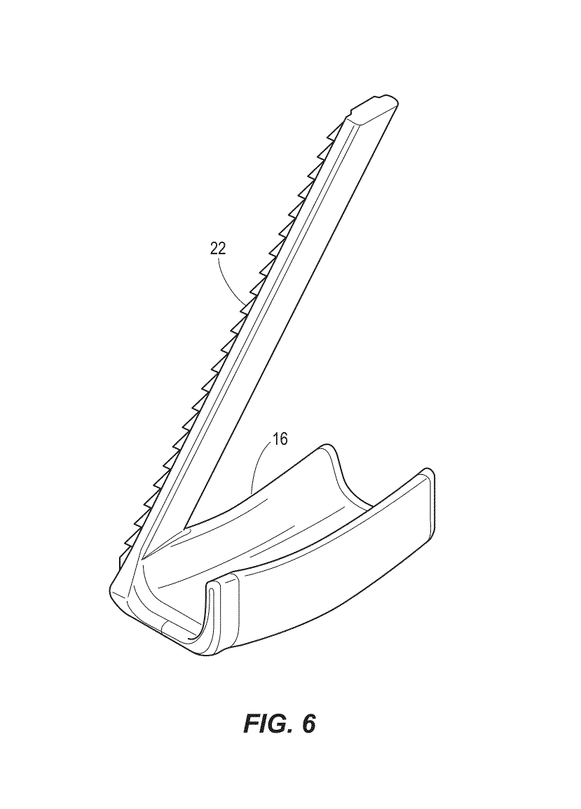

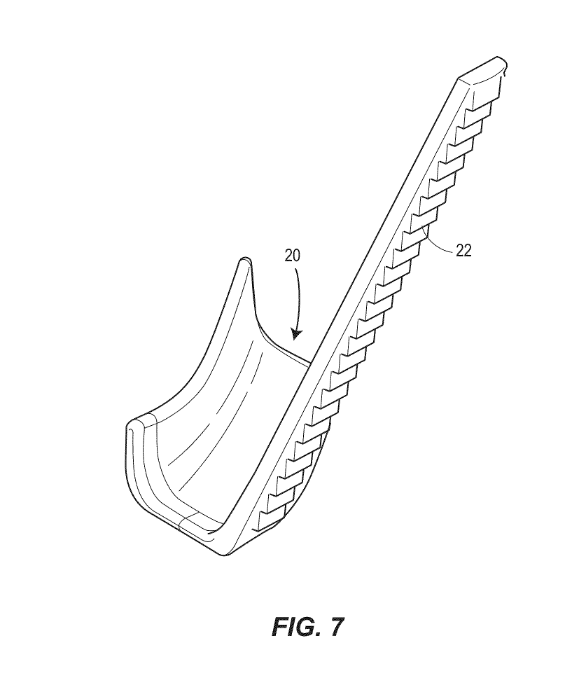

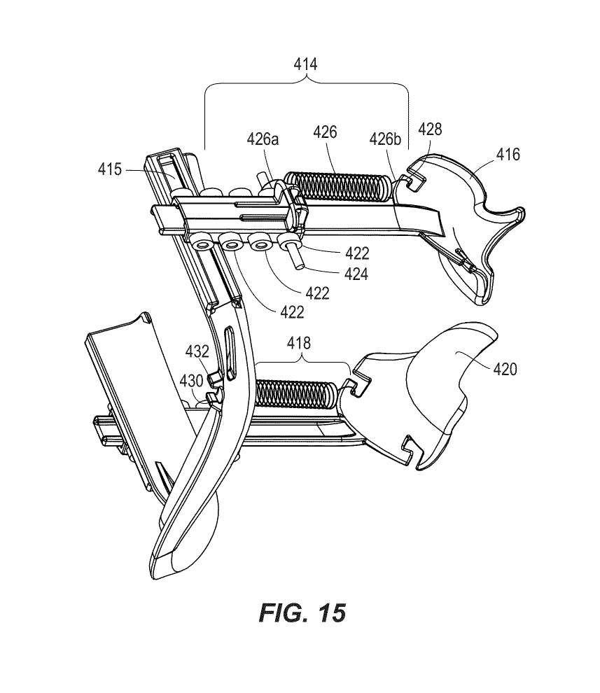

The airway support appliance 10 effectively provides a continuous jaw thrust by contacting the angle and ramus of each side of the mobile mandible, using the left and right jaw engagement pieces 16, 20. The left hinge and reversible tension mechanism 14 slidingly receives the left jaw engagement piece 16 and the right hinge and reversible tension mechanism 18 slidingly receives the right jaw engagement piece 20. The sliding engagement preferably includes a ratchet mechanism, such as a gear rack 22 (which is preferably provided as an integral extension of each of the left and right jaw engagement pieces 16, 20) and ratchet 24 (which is preferably integral with each of the left and right reversible tensioning mechanisms 14b, 18b), similar to a zip tie or cable tie, as can be seen in FIGS. 6 and 7. Each of the ratchet mechanisms is preferably releasable, such as by depressing a release wing 25 at a free end of the ratchet 24. Alternately, an adjustable spring mechanism (see FIG. 15) may be employed.

Each of the left and right hinge and reversible tension mechanisms 14, 18 include a pintle or hinge pin 26 that is received in one of two generally cylindrical connecting ends 28 of the bridge support member 12, one of which is disposed at either end of the bridge support member 12, such that the left and right hinge and reversible tension mechanisms 14, 18 rotate relative to the bridge support member 12. The hinge pin 26 may be a unitary member, but to facilitate assembly of the airway support appliance 10, the hinge pin 26 may instead comprise a first hinge pin portion and a second hinge pin portion separated by a gap. Tortional springs may be included at the connecting ends 28 of the bridge support member 12 to apply inward pressure between the respective jaw engagement pieces and the angle and ramus of each side of the mobile mandible.

In this embodiment, the bridge of the nose 15 of the patient is used as the stabilizing point. The bridge support member 12 is contoured in a dovetail shape to generally follow the contour of a patient's nose 15 and cheeks 17 so as to rest comfortably on the face when in use. The bridge support member is also of a low profile so as not to present an obstruction, for example, to oxygen tubes used to feed oxygen to the nasal or oral passages during a procedure involving sedation of the patient.

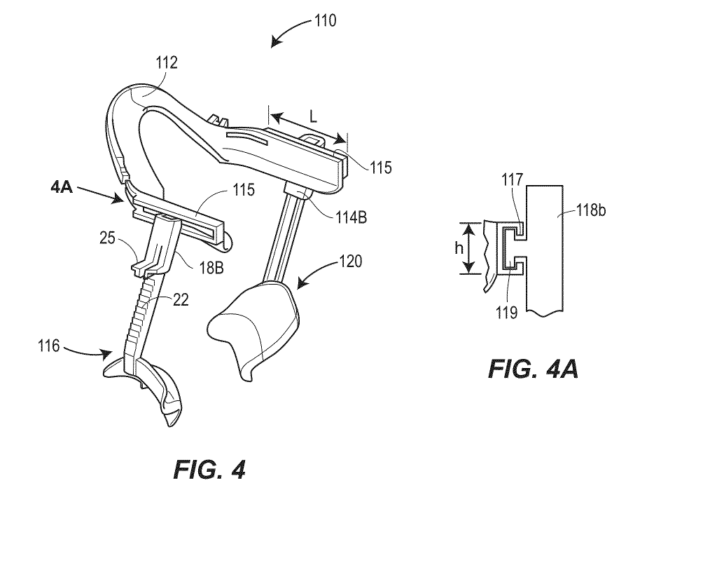

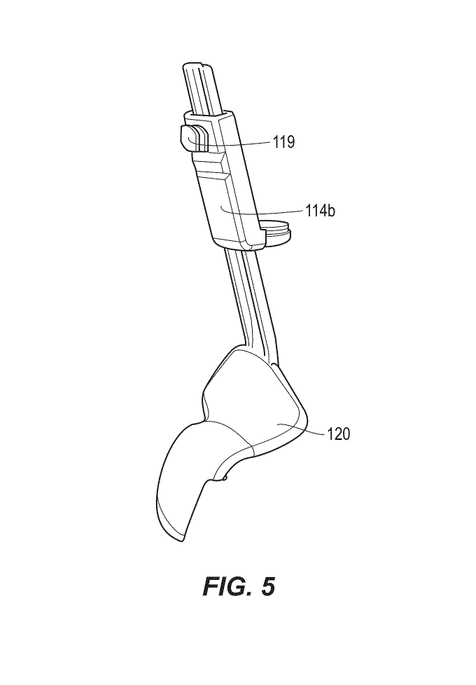

Turning to FIG. 4, an alternate embodiment of an airway support appliance 110 of the present disclosure is illustrated. Rather than a hinged connection, each of the left and right jaw engagement pieces 116, 120 is carried on a respective reversible tension mechanism 114, 118 that rides along a respective track member 115. The female receiving end 118b of the reversible tension mechanism includes a knob or engagement lug that can be slideably received in the track member 115. As illustrated in FIG. 4A, the track member 115 has a generally T-shaped opening 117, sized to receive the engagement lug 119 extending from the female receiving end 118b of the reversible tension mechanism. As best illustrated in FIG. 5, the engagement lug 119 is preferably oblong or asymmetrical (e.g., lemon-shaped), having a first dimension in a plane defined by the travel length L and the height h of the track member 115 that is less than the height h and another dimension in that same plane that is greater than or at least equal to the height h, such that in at least one orientation, the engagement lug 119 moves freely along the length of the track member 115, but when the reversible tension mechanism is rotated such that the engagement lug 119 is brought to another orientation, the lug resists movement along the length of the track member 115.

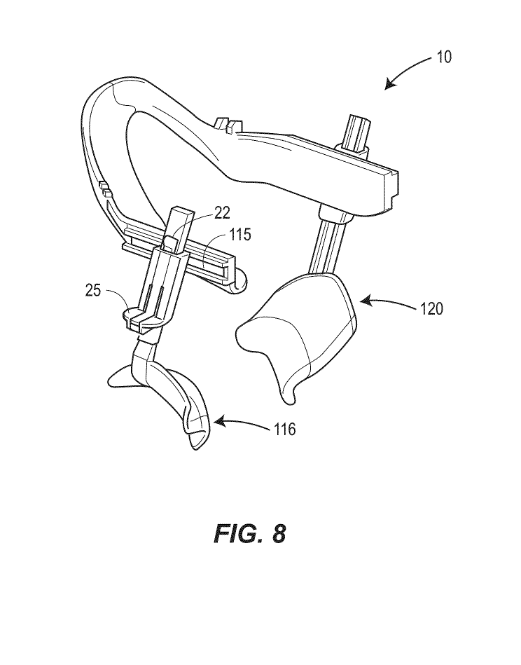

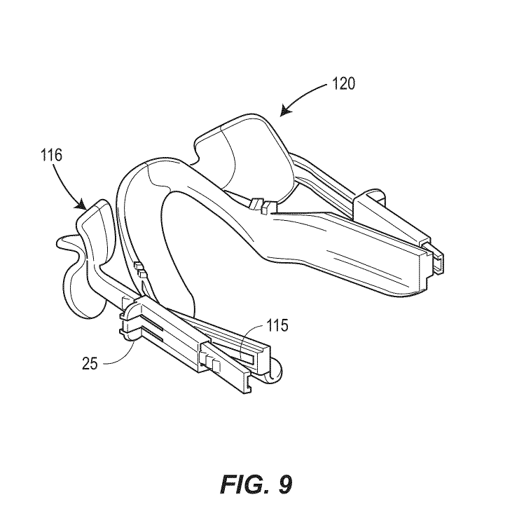

As illustrated in FIGS. 8 and 9, the engagement lugs 119 also permit the rotation of each of the reversible tension mechanisms 114, 118, and thereby, the respective left and right jaw engagement pieces 116, 120, relative to the track members 115. Rotation relative to the track members 115 is desirable so as to render the device more compact for packaging, storage (such as in a paramedic's pack, an ambulance, a medi-vac helicopter, or for positioning on a prep tray in an operating theater). Also, the reversible tension mechanisms 114, 118 of the left and right jaw-thrusting engagement pieces 116, 120 are advanced (i.e., shortened) relative to their positions illustrated in FIG. 4, so as to locate the engagement surfaces of the left and right jaw-thrusting engagement pieces 116, 120 closer to the bridge support member 112 of the airway support appliance.

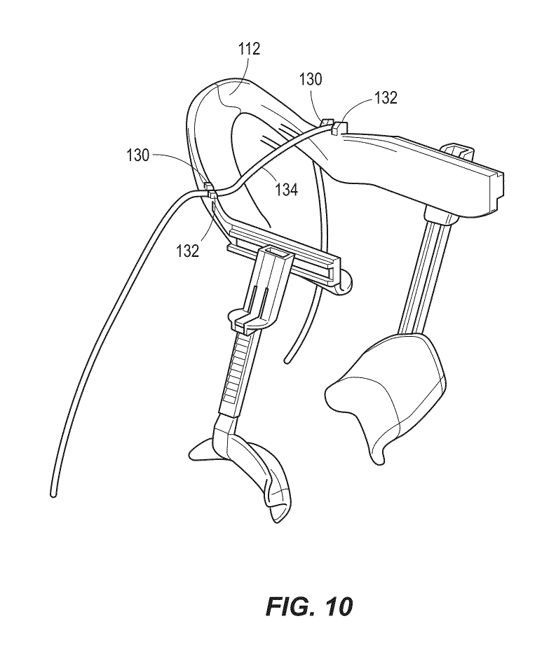

As best illustrated in FIG. 10, the bridge support member 112 may be provided with pairs of cleats 130, 132 to secure a nasal oxygen tube 134 to the airway support appliance 110.

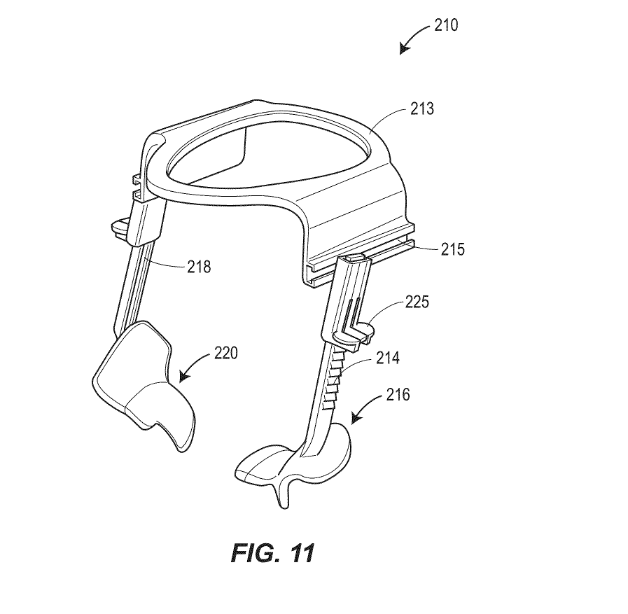

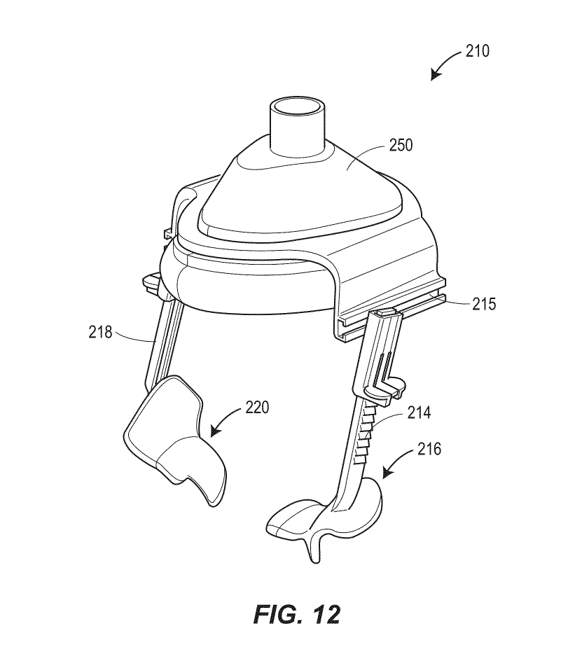

In yet another embodiment, illustrated in FIGS. 11 and 12, an airway support appliance 210 is capable of accepting a mask 250, such as an anesthesia mask as a part of BVM ventilation circuit, a continuous positive airway pressure (CPAP) or bilevel positive airway pressure (BiPAP) mask for use in noninvasive ventilation in emergencies or for sleep apnea. The device may also have attachments (not shown) that extend into the anterior part of the mouth to prevent occlusion of the mouth by keeping the upper and lower anterior teeth separated. In this embodiment, the airway support appliance 210 includes a frame 213 defining a mask-receiving aperture sized and shaped to receive, for example, an anesthesia mask 250 or a ventilation mask.

Like the airway support appliances of the other embodiments, the airway support appliance 210 of this embodiment includes left and right jaw engagement pieces 216, 220 movable along respective tracks 215, with reversible tension mechanisms 214, 218 operable via a release wing 225.

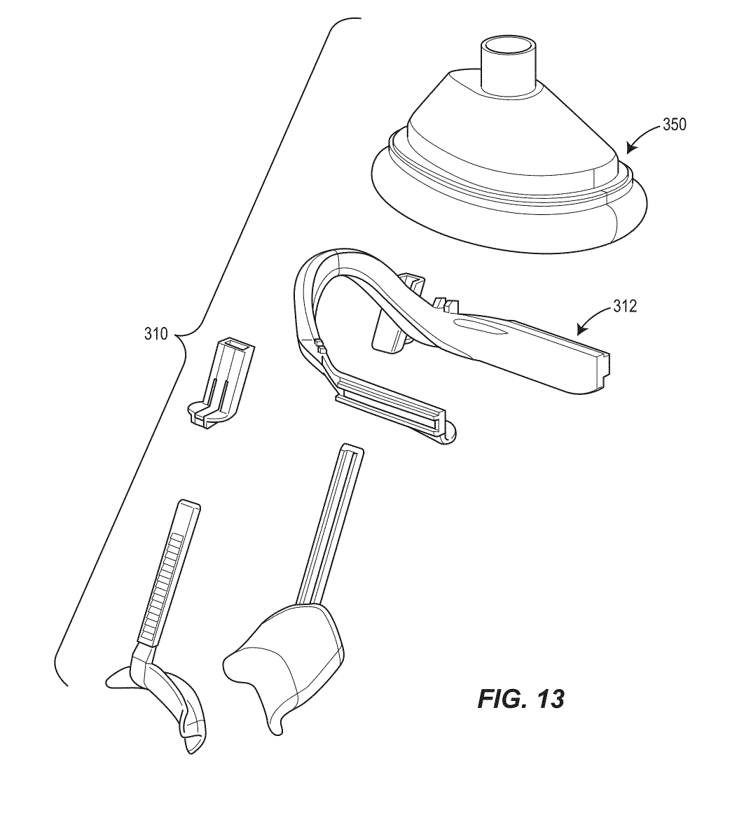

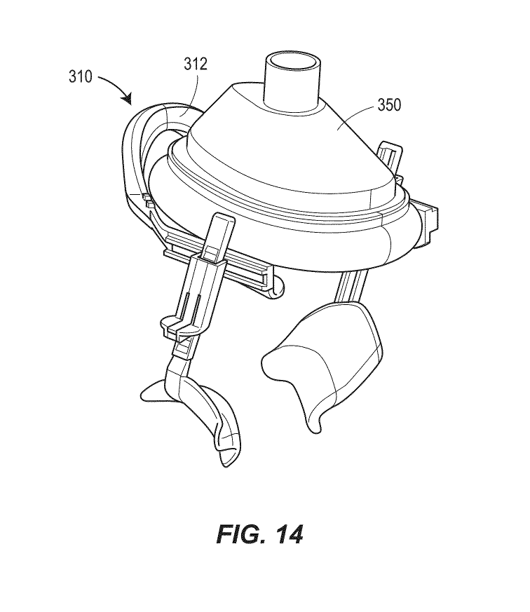

In yet a further embodiment, illustrated in FIGS. 13 and 14, an airway support appliance 310 is provided that is similar to that of FIG. 4, but the bridge support member 312 is provided with sufficient clearance to accommodate a standard anesthesia mask 350 or ventilation mask between the bridge support member 312 and the patient's face.

In an even further embodiment, illustrated in FIG. 15, an airway support appliance 410 is provided in which the ratchet-like reversible tension mechanisms, such as 114, 118, of the embodiments described above is replaced with spring mechanisms 414, 418. These spring mechanisms serve to bias the left and right jaw engagement pieces 416, 420 anteriorly when engaged against the angle and ramus of each side of the mobile mandible. The spring mechanisms 414, 418 preferably include a plurality of pin-receiving apertures 422 toward a track-engaging end, at least one of which receives a pin 424 to which a first end 426a of a spring 426 is secured. A second end 426b of the spring 426 is secured to a hook 428 provided on a respective one of the jaw engagement pieces 416, 420. The airway support appliance 410 further includes a pair of tracks 415 along which the spring mechanisms 414, 418 of the left and right jaw engagement pieces 416, 420 can translate, and within which they can rotate.

Separate cleats 430, 432 may be provided in the support member 412 to accommodate a nasal oxygen tube, with the apertures facilitating fluid (specifically, gaseous) communication via the oxygen tubes between a controlled oxygen source and the patient's nasal passages. Instead or in addition, the airway support appliance 410 (or of any of the embodiments disclosed herein) may include a plurality of interfaces permitting incorporation one or more sensors, each of the one or more sensors being selected from the group of a carbon dioxide sensor, a pulse oximetry sensor, an electro-cardiogram (ECG) sensor, a pulse sensor, a pressure sensitive sensor, a tissue perfusion sensor, a respiratory-effort sensor, and a thermometer. While the sensor-accommodating interface(s) may be provided along the support member 412, they may instead, or in addition, be provided in some other component of the airway support appliance 410, such as the left and/or right reversible tension mechanisms.

While various embodiments are disclosed, variations may be made thereto that are still within the scope of the appended claims.

What is claimed is:

1. An airway support appliance to reduce or prevent airway obstruction comprising:
a left jaw engagement member;
a left reversible tension mechanism longitudinally adjustable relative to the left jaw engagement member;
a right jaw engagement member;
a right reversible tension mechanism longitudinally adjustable relative to the right jaw engagement member; and
a support member having a first end engaged with the left reversible tension mechanism and a second end engaged with the right reversible tension mechanism.

2. The airway support appliance of claim 1, wherein at least one of the left and right reversible tension mechanisms includes a ratchet and the respective jaw engagement member includes a gear rack.

3. The airway support device of claim 1, wherein at least one of the left and right hinge and reversible tension mechanisms includes a hinge pin hingedly captured in a respective generally cylindrical end of the support member.

4. The airway support device of claim 1, wherein the support member is shaped so as to span a region between a nose and an upper lip of a patient.

5. The airway support device of claim 1, wherein the support member is shaped so as to span a region over the bridge of the nose of a patient.

6. The airway support device of claim 1, wherein the support member is shaped and sized so as to accommodate an anesthesia or ventilation mask.

7. The airway support device of claim 1, wherein at least one of the left and right reversible tension mechanisms includes a spring biasing the respective jaw engagement member toward the support member.

8. The airway support device of claim 1, wherein the device includes a plurality of interfaces permitting incorporation of flow of oxygen to nasal or oral passages.

9. The airway support device of claim 8, wherein the interfaces include cleats on the support member to secure a nasal oxygen tube to the support member.

10. The airway support device of claim 1, wherein the device includes a plurality of interfaces permitting incorporation one or more sensors, said one or more sensors being selected from the group of a carbon dioxide sensor, a pulse oximetry sensor, an electro-cardiogram (ECG) sensor, a pulse sensor, a pressure sensitive sensor, a tissue perfusion sensor, a respiratory-effort sensor, and a thermometer.

11. The airway support device of claim 1, including means for accepting one or more of:
a mask to facilitate BVM ventilation,
a continuous positive airway pressure (CPAP) mask, or
a bilevel positive airway pressure (BiPAP) mask for use in noninvasive ventilation.

12. The airway support device of claim 11, wherein the means includes a support member having an aperture sized and shaped to receive a mask.

13. The airway support device of claim 11, wherein the means includes a support member having a sufficient clearance to receive a mask between the support member and a face of a patient wearing the airway support device.
